(19) United States
(12) Patent Application Publication
(10) Pub. No.: US 2020/0054261 A1
(43) Pub. Date: Feb. 20, 2020

(54) MENTAL STRESS DETECTION DEVICE AND COMPUTER READABLE MEDIUM

(71) Applicant: Mitsubishi Electric Corporation, Tokyo (JP)

(72) Inventor: Mitsunari UOZUMI, Tokyo (JP)

(73) Assignee: Mitsubishi Electric Corporation, Tokyo (JP)

(21) Appl. No.: 16/341,377

(22) PCT Filed: Dec. 1, 2016
(86) PCT No.: PCT/JP2016/085773

§ 371 Date: Apr. 11, 2019

(51) Int. Cl. A61B 5/16; A61B 5/00; A61B 5/024; G06F 3/01; G06F 17/18

(52) U.S. Cl. CPC A61B 5/165; A61B 5/7246; A61B 5/7257; G06F 2203/011; A61B 5/4035; G06F 3/015; G06F 17/18; A61B 5/024

(57) ABSTRACT

A mental stress detection device (10) includes an index value calculation unit (200) and a correlation calculation unit (300). The index value calculation unit (200) calculates standard deviation (SDₙ) of heartbeat intervals (RRIₙ), a root mean square (RMₙ) of a difference RDₙ between temporally-adjacent heartbeat intervals (RRIₙ), and a ratio (SDₙ/RMₙ) between the standard deviation (SDₙ) and the root mean square (RMₙ). The root mean square (RMₙ) correlates with an activity of parasympathetic nerves and the ratio (SDₙ/RMₙ) correlates with an activity of sympathetic nerves. The correlation calculation unit (300) calculates a moment correlation coefficient (rₙ) which is a correlation between the root mean square (RMₙ) and (the ratio SDₙ/RMₙ) and a correlation associated with time.

### TECHNICAL FIELD

The present invention relates to a detection device and a detection program for detecting mental stress.

### BACKGROUND ART

Detection of mental stress has been conventionally performed such that the Fourier transformation is performed with respect to variation of a heartbeat interval so as to grasp activities of parasympathetic nerves and sympathetic nerves based on the obtained power spectrum and mental stress is estimated based on, for example, a table for converting HF power and a ratio between LF power and the HF power into stress indices (Patent Literature 1, for example).

### CITATION LIST

### Patent Literature

Patent Literature 1: JP 2007-167091A

### SUMMARY OF INVENTION

### Technical Problem

Conventional mental stress detectors are capable of evaluating stress only in a several-minute interval due to the employment of the Fourier transformation and accordingly, there has been a problem in that the conventional mental stress detectors cannot follow variation of mental stress which varies in seconds.

Further, stress caused by physical movement also decreases an activity level of parasympathetic nerves and increases an activity level of sympathetic nerves. However, an activity state of parasympathetic nerves and an activity state of sympathetic nerves have been independently observed in the prior art and accordingly, there has been a further problem in that mental stress caused by physical movement cannot be determined.

An object of the present invention is to provide a device that is capable of following variation of mental stress, which varies in seconds, and determining mental stress caused by physical movement.

### Solution to Problem

A mental stress detection device according to one aspect of the present invention includes:

an index value calculation unit to calculate a first index value, the first index value being an index of an activity state of parasympathetic nerves with elapse of time, and a second index value, the second index value being an index of an activity state of sympathetic nerves with elapse of time, based on a plurality of heartbeat intervals RRI; and

a correlation calculation unit to calculate a time corresponding correlation, the time corresponding correlation being a correlation between the first index value and the second index value and being a correlation associated with time.

### Advantageous Effects of Invention

The mental stress detection device according to the present invention includes the correlation calculation unit and accordingly, an object of the present invention is to provide a device that is capable of following variation of mental stress, which varies in seconds, and determining mental stress caused by physical movement.

### BRIEF DESCRIPTION OF DRAWINGS

FIG. 1 is a diagram of Embodiment 1 illustrating the hardware configuration of a mental stress detection device 10.

FIG. 2 is a diagram of Embodiment 1 illustrating the software configuration of the mental stress detection device 10.

FIG. 3 is a diagram of Embodiment 1 and a flowchart illustrating a first half of an operation of the mental stress detection device 10.

FIG. 4 is a diagram of Embodiment 1 and a flowchart illustrating a second half of the operation of the mental stress detection device 10.

FIG. 5 is a diagram of Embodiment 1 illustrating calculation of standard deviation SDₙ and a root mean square RMₙ.

FIG. 6 is a diagram of Embodiment 1 illustrating results detected by the mental stress detection device 10 in a graph.

FIG. 7 is a diagram of Embodiment 1 illustrating events in which a correlation coefficient rose in the graph of FIG. 6.

FIG. 8 is a diagram of Embodiment 1 illustrating a modification of the hardware configuration of the mental stress detection device 10.

### DESCRIPTION OF EMBODIMENTS

### Embodiment 1

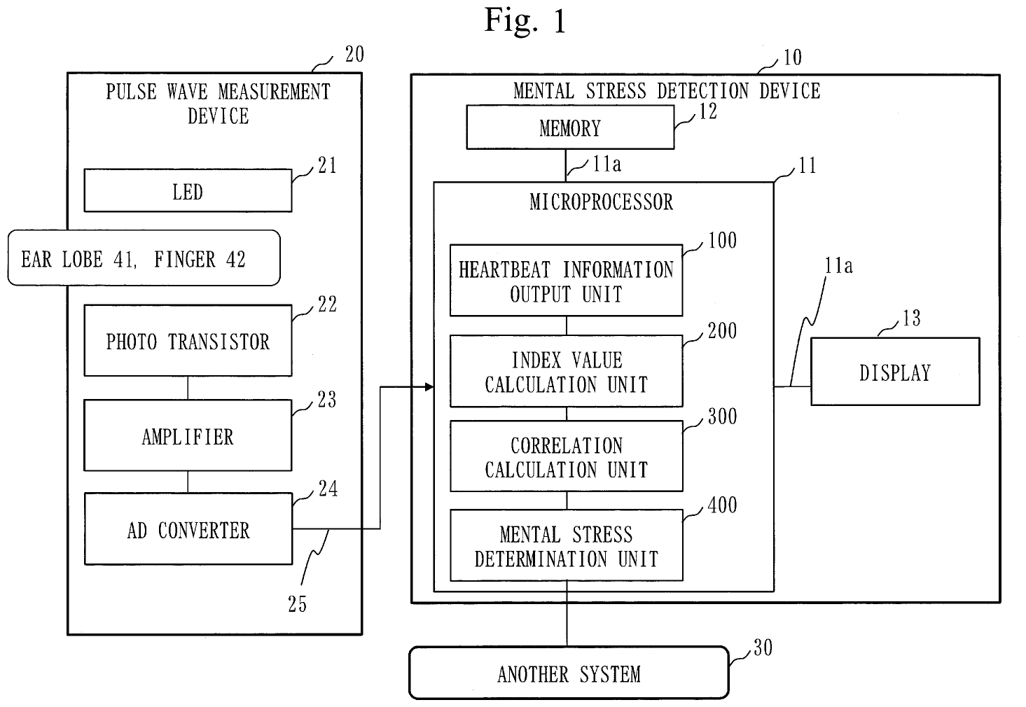

A mental stress detection device 10 is described with reference to FIG. 1 to FIG. 8.

***Description of Configuration***

FIG. 1 illustrates the hardware configurations of the mental stress detection device 10 and a pulse wave measurement device 20. The mental stress detection device 10 detects mental stress based on a waveform of a pulse wave acquired as a pulse wave signal 25 from the pulse wave measurement device 20. The hardware configuration of the mental stress detection device 10 is described with reference to FIG. 1.

The mental stress detection device 10 is a computer. The mental stress detection device 10 includes hardware such as a microprocessor 11, a memory 12, and a display 13. The microprocessor 11 is connected to other pieces of hardware via a signal line 11a and controls these other pieces of hardware.

The microprocessor 11 is an IC (Integrated Circuit) which performs arithmetic operations. Specific examples of the microprocessor 11 include a CPU (Central Processing Unit), a DSP (Digital Signal Processor), and a GPU (Graphics Processing Unit).

The memory 12 stores a program for realizing the function of the mental stress detection device 10, data generated by the microprocessor 11, and data inputted into the mental stress detection device 10. Specific examples of the memory 12 include a HDD (Hard Disk Drive), an SD (Secure Digital) memory card, a CF (Compact Flash), a NAND flash, a flexible disk, an optical disk, a compact disk, and a DVD (Digital Versatile Disk). The memory 12 may be a portable storage medium.

The display 13 is controlled by the microprocessor 11. When the microprocessor 11 detects rise of mental stress, the microprocessor 11 displays the detection on the display 13.

The mental stress detection device 10 includes a heartbeat information output unit 100, an index value calculation unit 200, a correlation calculation unit 300, and a mental stress determination unit 400 as functional components. Functions of the heartbeat information output unit 100, the index value calculation unit 200, the correlation calculation unit 300, and the mental stress determination unit 400 are realized by software. A program for realizing the functions of the heartbeat information output unit 100, the index value calculation unit 200, the correlation calculation unit 300, and the mental stress determination unit 400 is stored in the memory 12. This program is read and executed by the microprocessor 11. Thus, the functions of the heartbeat information output unit 100, the index value calculation unit 200, the correlation calculation unit 300, and the mental stress determination unit 400 are realized.

FIG. 1 illustrates only one microprocessor 11. However, the mental stress detection device 10 may include a plurality of processors substituting for the microprocessor 11. These plurality of processors execute the program for realizing the functions of the heartbeat information output unit 100, the index value calculation unit 200, the correlation calculation unit 300, and the mental stress determination unit 400 in a sharing manner. Each of the processors is an IC performing arithmetic operations as is the case with the microprocessor 11.

The pulse wave measurement device 20 measures a pulse wave from an ear lobe 41 or a finger 42 of a person. An LED 21 emits an infrared ray, for example, and a photo transistor 22 detects variation in blood flow. An amplifier 23 amplifies an output of the photo transistor 22. An AD converter 24 is an AD (Analog to digital) converter that converts an analog signal which is an output of the amplifier 23 into a digital signal and outputs the digital signal as the pulse wave signal 25 to the mental stress detection device 10. The pulse wave signal 25 is a signal indicating variation in blood flow. A peak of blood flow is a peak of a pulse wave. Further, a peak of a pulse wave corresponds to a heartbeat and peak time of a pulse wave is heartbeat time.

Another system 30 receives mental stress detected by the mental stress detection device 10 as a signal and performs logging or the like.

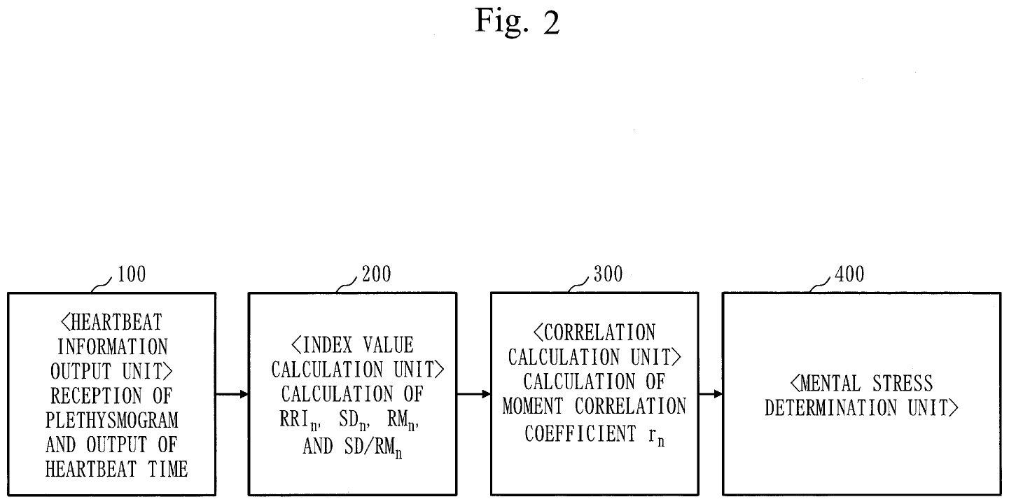

FIG. 2 illustrates the software configuration of the mental stress detection device 10.

The heartbeat information output unit 100 receives the pulse wave signal 25 indicating a plethysmogram from the pulse wave measurement device 20 and outputs heartbeat time. The heartbeat information output unit 100 receives the pulse wave signal 25 outputted from the AD converter 24 of the pulse wave measurement device 20 and calculates time Rₙ at which a peak of a pulse wave comes. The time Rₙ which is peak time of a pulse wave is also time of a heartbeat. The time Rₙ is referred to below as heartbeat time.

The index value calculation unit 200 calculates an heartbeat interval RRIₙ which is an interval between the heartbeat time Rₙ and heartbeat time Rₙ₋₁ (RRIₙ=Rₙ−Rₙ₋₁ described later), a standard deviation SDₙ which is standard deviation of the heartbeat interval RRIₙ, a root mean square RMₙ which is a root mean square of differences between adjacent heartbeat intervals RRIₙ (RDₙ=|RRIₙ−RRIₙ₋₁| described later), and a ratio SDₙ/RMₙ which is a ratio between the standard deviation SDₙ and the root mean square RMₙ. Here, the ratio SDₙ/RMₙ is sometimes referred to as SD/RMₙ or SDRMₙ below. The heartbeat interval RRIₙ, the standard deviation SDₙ, the root mean square RMₙ, and the ratio SDₙ/RMₙ are described later.

The correlation calculation unit 300 calculates a moment correlation coefficient rₙ on the root mean square RMₙ and the ratio SD/RMₙ outputted from the index value calculation unit 200.

The mental stress determination unit 400 determines the moment correlation coefficient rₙ outputted from the correlation calculation unit 300, and when the mental stress determination unit 400 determines that mental stress is high, the mental stress determination unit 400 performs lighting of the display 13 and notification to another system 30.

***Description of Operation***

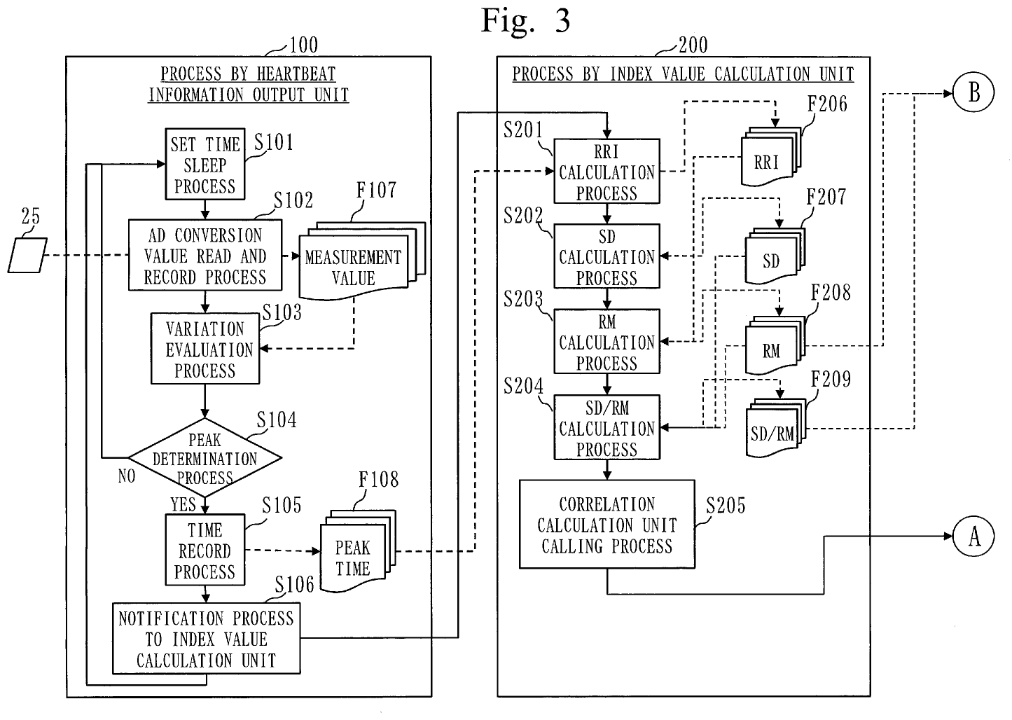

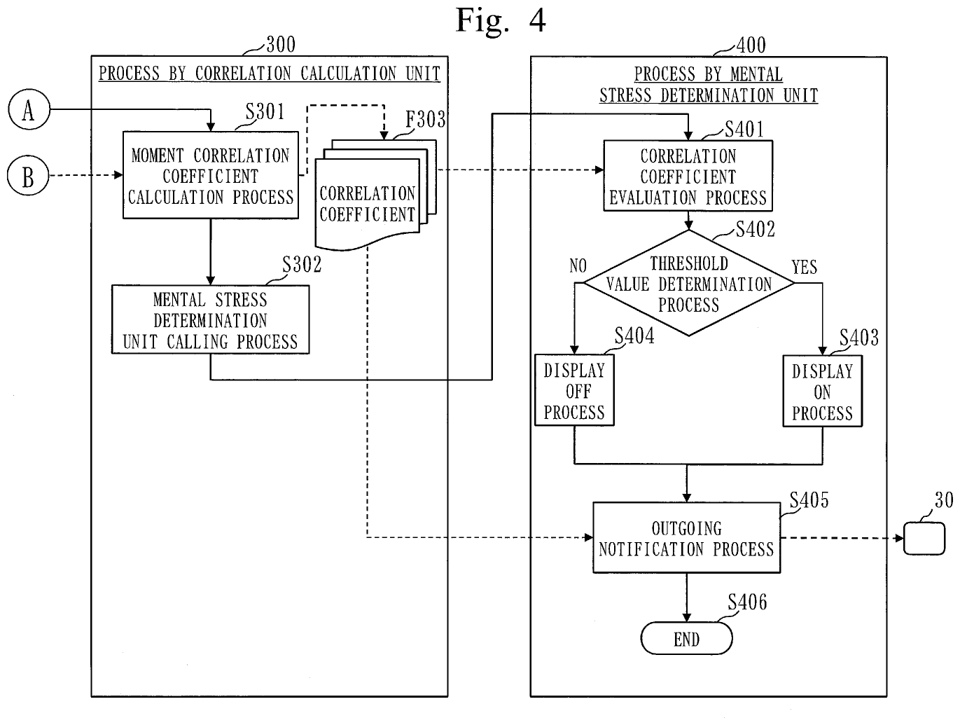

FIG. 3 and FIG. 4 are flowcharts illustrating the operation of the mental stress detection device 10.

FIG. 3 is the flowchart illustrating the first half in the operation of the mental stress detection device 10.

FIG. 4 is the flowchart illustrating the second half in the operation of the mental stress detection device 10.

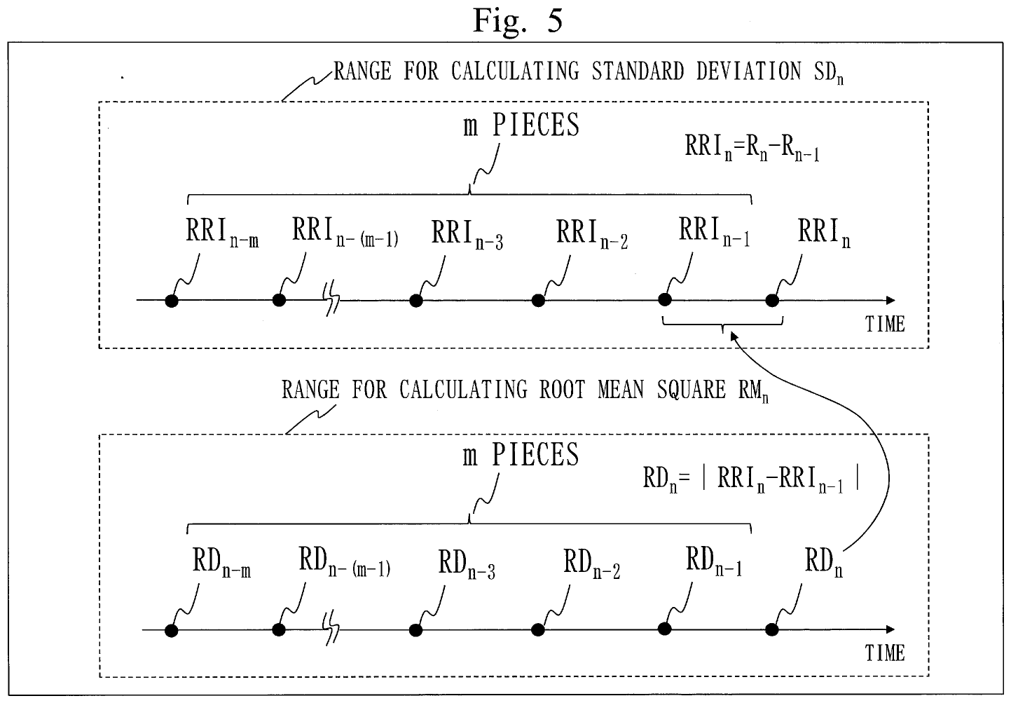

FIG. 5 is a diagram illustrating calculation of the standard deviation SDₙ and the root mean square RMₙ.

An outline of the operation of the mental stress detection device 10 is described with reference to FIG. 3 and FIG. 4. The operation of the mental stress detection device 10 corresponds to a mental stress detection method. Further, the operation of the mental stress detection device 10 corresponds to a process of a mental stress detection program. Frames in FIG. 3 and FIG. 4 each drawn for the heartbeat information output unit 100, the index value calculation unit 200, the correlation calculation unit 300, and the mental stress determination unit 400 show processes executed by the heartbeat information output unit 100 and the others. A process executed by the heartbeat information output unit 100 is denoted by a reference character provided with S as a set time sleep process (S101) and data to be written in a file by the heartbeat information output unit 100 is denoted by a reference character provided with F as a measurement value file (F107). The same applies to those of the index value calculation unit 200, the correlation calculation unit 300, and the rest. Writing in a file means writing in the memory 12.

The pulse wave measurement device 20 is attached to the ear lobe 41 or the finger 42 of a subject. In the following description, it is assumed that the pulse wave measurement device 20 is attached to the ear lobe 41 of a subject. The LED 21 and the photo transistor 22 pinch the ear lobe 41 and the photo transistor 22 catches variation in blood flow of the subject. The amplifier 23 amplifies an output of the photo transistor 22 and the AD converter 24 converts an analog signal outputted from the amplifier 23 into a digital signal. This digital signal is inputted into the microprocessor 11 as the pulse wave signal 25. Mental stress is evaluated by the functions of the heartbeat information output unit 100, the index value calculation unit 200, the correlation calculation unit 300, and the mental stress determination unit 400 executed by the microprocessor 11 in software. Display by the display 13 and notification to another system 30 are performed depending on an evaluation result of mental stress.

An outline of the operations of the heartbeat information output unit 100, the index value calculation unit 200, the correlation calculation unit 300, and the mental stress determination unit 400 is described.

(1) The heartbeat information output unit 100 first detects a peak of a pulse wave from the pulse wave signal 25 which is an output of the AD converter 24 and records occurrence time of the peak.

(2) The index value calculation unit 200 calculates the heartbeat interval RRIₙ which is a peak interval, the standard deviation SDₙ of the heartbeat interval RRIₙ, the root mean square RMₙ of a difference RDₙ between adjacent heartbeat intervals RRIₙ and RRIₙ₋₁, and the ratio SDₙ/RMₙ, which is a ratio between the standard deviation SDₙ and the root mean square RMₙ, in response to notification from the heartbeat information output unit 100 at the time of the peak detection. A section for calculating the standard deviation SDₙ of the heartbeat interval RRIₙ, and the root mean square RMₙ of the difference RDₙ between adjacent heartbeat intervals is in a range of last in pieces from the latest peak, but the range of approximately m=20 pieces is appropriate. The description of m pieces is given when FIG. 5 is referred to.

(3) The correlation calculation unit 300 is called by the index value calculation unit 200 and calculates the moment correlation coefficient rₙ between the root mean square RMₙ and the ratio SDₙ/RMₙ.

(4) The mental stress determination unit 400 is called by the correlation calculation unit 300 and evaluates the moment correlation coefficient rₙ. The moment correlation coefficient rₙ has a value within a range from −1.0 to +1.0 and the moment correlation coefficient rₙ is distinguished based on a threshold value. The threshold value is a preset value. The mental stress determination unit 400 determines that mental stress is high when the moment correlation coefficient rₙ exceeds the threshold value. An appropriate threshold value is approximately −0.2. When mental stress is high, the mental stress determination unit 400 displays the display 13. Further, the mental stress determination unit 400 transmits the moment correlation coefficient rₙ to another system 30.

The index value calculation unit 200, the correlation calculation unit 300, and the mental stress determination unit 400 are executed every peak detection by the heartbeat information output unit 100 in response to notification from the heartbeat information output unit 100, that is, notification through a notification process to index value calculation unit (S106). However, the index value calculation unit 200 and the others may be not only executed based on the notification process to index value calculation unit (S106) but also be processes or threads independent from the heartbeat information output unit 100. Alternatively, the index value calculation unit 200 and the others may be executed as subroutines of the heartbeat information output unit 100.

The operations of the heartbeat information output unit 100 and the others are described in detail below. The heartbeat information output unit 100 is periodically operated based on the set time sleep process (S101) so as to evaluate the pulse wave signal 25 which is outputted by the AD converter 24 in accordance with a sampling cycle. In FIG. 3, the pulse wave signal 25 outputted by the AD converter 24 is referred to at a set sampling cycle in the set time sleep process (S101). The sampling cycle of the set time sleep process (S101) is approximately from 500 Hz to 1000 Hz.

An AD conversion value read and record process (S102) is periodically executed and the pulse wave signal 25 is read and recorded in the measurement value file (F107) every execution of the AD conversion value read and record process (S102).

In a variation evaluation process (S103), a measurement value recorded in the measurement value file (F107) is referred to and evaluated. The pulse wave signal 25 is evaluated based on a threshold value and a differential value of the pulse wave signal 25 in the variation evaluation process (S103). The variation evaluation process (S103) is executed every time the pulse wave signal 25 is read in, and the measurement value file (F107) is referred to. Variation is evaluated based on an arbitrary algorithm in the variation evaluation process (S103).

In a peak determination process (S104), whether or not to be a peak of the pulse wave signal 25 is determined based on a result of the evaluation in the variation evaluation process (S103). A time record process (S105) is executed when a peak of the pulse wave signal 25 is determined in the peak determination process (S104). On the other hand, when a peak is not determined, a sleep state starts by the set time sleep process (S101) and continues until the following sampling cycle. When a peak is determined in the peak determination process (S104), peak time is recorded in a peak time file (F108) through the time record process (S105) and the index value calculation unit 200 is notified of the detection of the peak of the pulse wave signal 25 through the notification process to index value calculation unit (S106). When a peak is not determined in the peak determination process (S104), control is returned from the notification process to index value calculation unit (S106) to the set time sleep process (S101). Accuracy in approximately 1/1000 seconds is suitable to determine peak time. A counter value per millisecond from boot of the microprocessor 11 may be employed as long as the counter value has accuracy in approximately 1/1000 seconds.

Time or a counter value from the boot of the microprocessor 11 is recorded in the peak time file (F108) in the time record process (S105). Further, the notification process to index value calculation unit (S106) is operated from the time record process (S105) and the index value calculation unit 200 is notified of the peak occurrence, and a sleep state starts and continues until the following sampling cycle. In an RRI calculation process (S201), the peak time file (F108) is referred to, a difference between the peak time Rₙ and the peak time Rₙ₋₁ immediately preceding the peak time Rₙ is obtained as the heartbeat interval RRIₙ, and the heartbeat interval RRIₙ is recorded in an RRI file (F206) which is a file for the heartbeat interval RRI. That is, the index value calculation unit 200 calculates the heartbeat interval RRI in the RRI calculation process (S201). Peak occurrence time is the heartbeat time Rₙ. When peak occurrence time, that is, certain heartbeat time is set as Rₙ, the heartbeat interval RRIₙ which is a difference between the heartbeat time Rₙ and the heartbeat time Rₙ₋₁ immediately preceding the heartbeat time Rₙ is expressed as expression 1.

RRIₙ=Rₙ−Rₙ₋₁ (1)

In an SD calculation process (S202), the standard deviation SD in a range of the last m pieces of heartbeat intervals RRI is obtained and is recorded in a SD file (F207) which is a file for the standard deviation SD. FIG. 5 illustrates an outline of calculation for the standard deviation SD of the last m pieces of heartbeat intervals RRI. The heartbeat interval RRIₙ represents a current heartbeat interval and the heartbeat interval RRIₙ₋₁ represents a heartbeat interval immediately preceding the heartbeat interval RRIₙ.

The standard deviation SDₙ of the heartbeat interval RRIₙ is calculated in the SD calculation process (S202). When calculation is performed for peaks of the last m pieces of pulse waves as illustrated in FIG. 5, the standard deviation SDₙ is expressed as expression 3. Expression 2 is an expression for obtaining an average of the heartbeat intervals RRI. More specifically, performing calculation for peaks of the last m pieces of pulse waves means that the standard deviation SDₙ, the root mean square RMₙ, and so on are calculated with respect to RRIₙ₋ₘ to RRIₙ in FIG. 5.

$$\begin{matrix}FORMULA 1 & \\ {\overline{RRI}}_{n} = \frac{1}{m} {\sum }_{i = n - m}^{n} {RRI}_{i} & ( 2 ) \\ {SD}_{n} = \sqrt{\frac{1}{m} {\sum }_{i = n - m}^{n} {( {RRI}_{i} - {\overline{RRI}}_{n} )}^{2}} & ( 3 )\end{matrix}$$

In an RM calculation process (S203), the root mean square RM is obtained for differences between adjacent RRIₙ in a range of the last m pieces and is recorded in an RM file (F208) which is a file for recording root mean squares RM. FIG. 5 illustrates an outline of calculation for the root mean square RM in the last m pieces, shown below the standard deviation SD. The root mean square RMₙ of differences between adjacent heartbeat intervals RRIₙ is obtained in the RM calculation process (S203). When calculation is performed for the last m pieces of peaks, the root mean square RMₙ is expressed as expression 5. Expression 4 is an expression for obtaining a difference RDₙ between adjacent heartbeat intervals RRIₙ.

$$\begin{matrix}FORMULA 2 & \\ {RD}_{n} = \vert {RRI}_{n} - {RRI}_{n - 1} \vert & ( 4 ) \\ FORMULA 3 & \\ {RM}_{n} = \sqrt{\frac{1}{m} {\sum }_{i = n - m}^{n} {( {RD}_{i} )}^{2}} & ( 5 )\end{matrix}$$

In an SD/RM calculation process (S204), the SD file (F207) and the RM file (F208) are referred to and the ratio SD/RM between SD and the RM at the same time is obtained and recorded in a SD/RM file (F209). In a correlation calculation unit calling process (S205), the correlation calculation unit 300 is called. Expression 6 is the ratio SD/RMₙ calculated in the SD/RM calculation process (S204) and representing a ratio between the standard deviation SDₙ and the root mean square RMₙ.

$$\begin{matrix}FORMULA 4 & \\ SD / {RM}_{n} = \frac{{SD}_{n}}{{RM}_{n}} & ( 6 )\end{matrix}$$

The root mean square RMₙ correlates to an activity of parasympathetic nerves and the ratio SD/RMₙ correlates to an activity of sympathetic nerves. The root mean square RMₙ is the first index value which is an index of an activity state of parasympathetic nerves with elapse of time. The ratio SD/RMₙ is the second index value which is an index of an activity state of sympathetic nerves with elapse of time.

In a moment correlation coefficient calculation process (S301), the RM file (F208) and the SD/RM file (F209) are referred to so as to calculate the moment correlation coefficient rₙ and the moment correlation coefficient rₙ is recorded in a correlation coefficient file (F303).

In a mental stress determination unit calling process (S302), the mental stress determination unit 400 is called.

The moment correlation coefficient calculation process (S301) is described in detail below. The correlation calculation unit 300 evaluates a correlation between the root mean square RMₙ and the ratio SD/RMₙ. The root mean square RMₙ and the ratio SD/RMₙ have a negative correlation in an ordinary condition of a person, while the negative correlation between the root mean square RMₙ and the ratio SD/RMₙ is lost when mental stress rises. A correlation between the root mean square RMₙ and the ratio SD/RMₙ in a set section Lₙ denoted as Lₙ in expression 9 is evaluated based on the moment correlation coefficient rₙ. The number of pieces of Lₙ is preferably from 20 to 30, but the number is not limited to this. When the section is set to in pieces of peaks, the moment correlation coefficient rₙ is expressed as expression 9. The moment correlation coefficient rₙ represents a correlation between the root mean square RMₙ which is the first index value and the ratio SD/RMₙ which is the second index value and represents a time corresponding correlation which is a correlation associated with time. A value of the moment correlation coefficient rₙ which is a time corresponding correlation is determined with respect to time. Expression 7 is an expression for obtaining an average of the root mean squares RMₙ. Expression 8 is an expression for obtaining an average of the ratios SD/RMₙ. Regarding each of expression 7, expression 8, and expression 9, i, m, n, and so forth in the expression of Σ are closed within the expression. In other words,

i, n, and m in expression 7 are used only in expression 7,

i, n, and m in expression 8 are used only in expression 8, and

i, n, and Lₙ in expression 9 are used only in expression 9.

$$\begin{matrix}FORMULA 5 & \\ {\overline{RM}}_{n} = \frac{1}{m} {\sum }_{i = n - m}^{n} {RM}_{i} & ( 7 ) \\ {\overline{SDRM}}_{n} = \frac{1}{m} {\sum }_{i = n - m}^{n} {SDRM}_{i} & ( 8 ) \\ {r}_{n} = \frac{{\sum }_{i = n - {L}_{n}}^{n} ( {RM}_{i} - {\overline{RM}}_{n} ) ( {SDRM}_{i} - {\overline{SDRM}}_{n} )}{\sqrt{{\sum }_{i = n - {L}_{n}}^{n} {( {RM}_{i} - {\overline{RM}}_{n} )}^{2} {\sum }_{i = n - {L}_{n}}^{n} {( {SDRM}_{i} - {\overline{SDRM}}_{n} )}^{2}}} & ( 9 )\end{matrix}$$

In a correlation coefficient evaluation process (S401), a threshold value and the moment correlation coefficient rₙ recorded in the correlation coefficient file (F303) are mutually compared and evaluated. The moment correlation coefficient rₙ is compared with the threshold value and whether or not mental stress of a person whose heartbeat Rₙ is measured has risen is determine through the comparison and determination, in the correlation coefficient evaluation process (S401). The threshold value used for determination in the correlation coefficient evaluation process (S401) is set to −0.2.

In the case of −0.2≤moment correlation coefficient rₙ,

it is determined that mental stress is high in the correlation coefficient evaluation process (S401).

In the case of −0.2≥moment correlation coefficient rₙ,

it is not determined that mental stress is high in the correlation coefficient evaluation process (S401).

In a threshold value determination process (S402), an evaluation result obtained through the correlation coefficient evaluation process (S401) is determined. That is, whether it is determined that mental stress is high or it is not determined that mental stress is high through the correlation coefficient evaluation process (S401) is confirmed in the threshold value determination process (S402).

The display 13 is turned ON in a display ON process (S403) if it is determined that mental stress is high in the threshold value determination process (S402). The display 13 is turned OFF in a display OFF process (S404) if it is not determined that mental stress is high. In an outgoing notification process (S405), another system 30 is notified of the determination result obtained through the threshold value determination process (S402).

Alternatively, another system 30 may be notified of data recorded in the correlation coefficient file (F303) in the outgoing notification process (S405). Then, another system 30 may execute the operation of the mental stress determination unit 400. That is, the mental stress determination unit 400 is an output unit which is capable of outputting at least one of a determination result obtained through the threshold value determination process (S402) and data of the correlation coefficient file (F303).

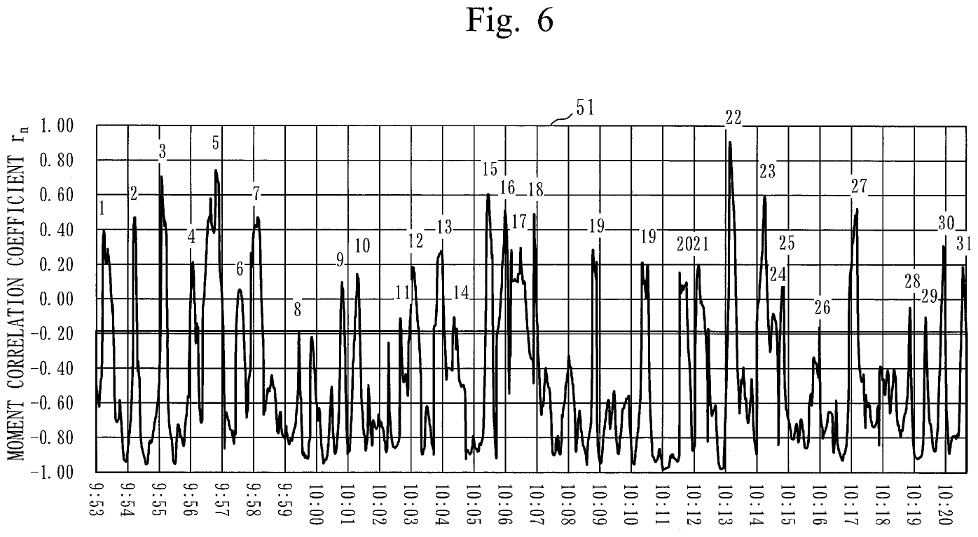

FIG. 6 illustrates results, which are obtained by logging in another system 30 when the mental stress detection device 10 is applied, in a graph.

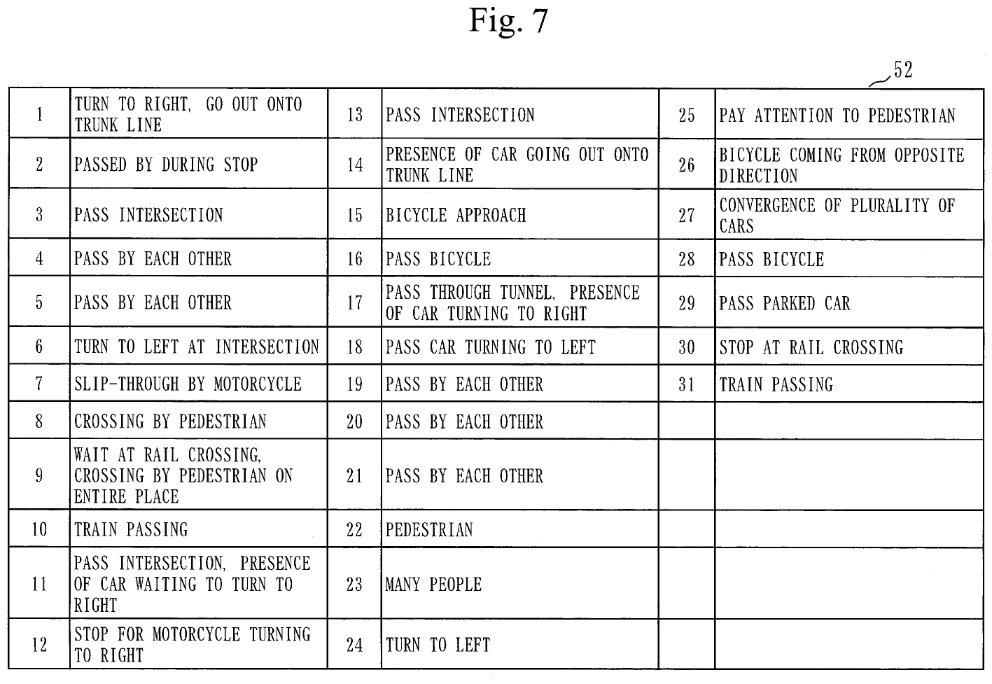

FIG. 7 is a diagram illustrating events in which a correlation coefficient rose in the graph of FIG. 6. The horizontal axis and the vertical axis of the graph 51 of FIG. 6 respectively represent time and the moment correlation coefficient rₙ. In terms of time, 9:53 on the left side represents time of nine fifty-three. The table 52 in FIG. 7 shows events in which a correlation coefficient rose. The graph 51 shows a case where data of the correlation coefficient file (F303) is outputted to another system 30 in the outgoing notification process (S405). FIG. 6 illustrates data acquired by another system 30 in a graph. Correlation can be seen between events in which the moment correlation coefficient rₙ rose in the graph 51 of FIG. 6 and events in driving in the table 52 of FIG. 7. Whether or not a driver has noticed the events can be determined based on the level of mental stress. Though the threshold value is set to −0.2 in the above-described example, the mental stress determination unit 400 may determine that a period in which the moment correlation coefficient rₙ does not show a negative correlation is a period in which mental stress is higher than in other periods.

In an end process (S406), the process from the RRI calculation process (S201) which is executed in response to the notification process to index value calculation unit (S106) is completed.

### Advantageous Effect of Embodiment 1

According to the mental stress detection device 10 of Embodiment 1, the correlation calculation unit 300 calculates the moment correlation coefficient rₙ which is a correlation between the root mean square RMₙ and the ratio SD/RMₙ. Accordingly, mental stress caused by physical movement can be determined.

Further, since the moment correlation coefficient rₙ calculated by the correlation calculation unit 300 is a time corresponding correlation which is associated with time, variation of mental stress which varies in seconds can be followed.

### Modification 1

Embodiment 1 described above has the configuration in which the heartbeat Rₙ is measured by the pulse wave measurement device 20, but the pulse wave measurement device 20 can be replaced by an electrocardiograph. However, in this case using an electrocardiograph, the number of probes attached on a subject is larger than the case where pulse waves are measured by the pulse wave measurement device 20. Further, it is sometimes difficult to detect the heartbeat interval RRIₙ unless an effect of myoelectricity caused by movement of an arm or the like is removed by a filter or the like.

### Modification 2

A DSP incorporated in the pulse wave measurement device 20 may perform the process up to the peak determination process (S104) of the heartbeat information output unit 100. In this case, the pulse wave signal 25 is not a value representing blood flow but is an interruption signal at peak detection timing, and the time record process (S105) is booted by an interruption process. This method does not require the microprocessor 11 to evaluate the pulse wave signal 25 every sampling cycle, being suitably applied to a microprocessor having low throughput.

### Modification 3

In the above-described embodiment, the configuration is described in which an activity level of parasympathetic nerves and an activity level of sympathetic nerves are respectively evaluated based on the root mean square RMₙ and the ratio SD/RMₙ. Alternatively, the configuration may be employed in which the Fourier transformation is performed with respect to a result of the RRI calculation process (S201) so as to derive activity levels of parasympathetic nerves and sympathetic nerves based on the frequency components and moment correlation coefficients of the two are calculated in the moment correlation coefficient calculation process (S301). However, sections of approximately hundreds times of heartbeat are required so as to obtain beneficial results from the Fourier transformation, so that this configuration is not suitable for grasping variation in activity levels of nerves of a person in short time.

<***Other Configuration***>

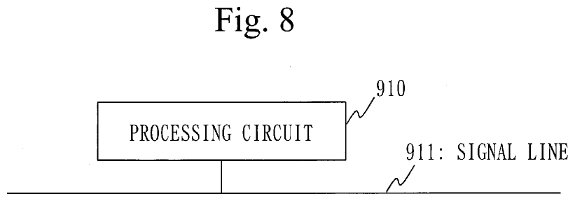

FIG. 8 is a diagram illustrating a processing circuit 910. The functions of the heartbeat information output unit 100, the index value calculation unit 200, the correlation calculation unit 300, and the mental stress determination unit 400 are realized by software in Embodiment 1. FIG. 8 is a diagram illustrating the processing circuit 910 as a modification. The functions of the heartbeat information output unit 100, the index value calculation unit 200, the correlation calculation unit 300, and the mental stress determination unit 400 may be realized by hardware in Embodiment 1. That is, the functions of the heartbeat information output unit 100, the index value calculation unit 200, the correlation calculation unit 300, and the mental stress determination unit 400 which are represented as the microprocessor 11 described above and the function of the memory 12 described above are realized by the processing circuit 910. The processing circuit 910 is connected to a signal line 911. The processing circuit 910 is an electronic circuit. Specifically, the processing circuit 910 is a single circuit, a composite circuit, a programmed processor, a parallel programmed processor, a logic IC, a GA (Gate Array), an ASIC (Application Specific Integrated Circuit), or a FPGA (Field-Programmable Gate Array).

As another modification, the functions of the heartbeat information output unit 100, the index value calculation unit 200, the correlation calculation unit 300, and the mental stress determination unit 400 may be realized by a combination of software and hardware. The microprocessor 11 and the processing circuit 910 are collectively called as a “processing circuitry”. The functions of the heartbeat information output unit 100, the index value calculation unit 200, the correlation calculation unit 300, and the mental stress determination unit 400 are realized by the processing circuitry. The operation of the mental stress detection device 10 may be considered as a mental stress detection program. Further, the operation of the mental stress detection device 10 may be considered as a mental stress detection method.

### REFERENCE SIGNS LIST

10: mental stress detection device; 11: microprocessor; 11a: signal line; 100: heartbeat information output unit; S101: set time sleep process; S102: AD conversion value read and record process; S103: variation evaluation process; S104: peak determination process; S105: time record process; S106: notification process to index value calculation unit; F107: measurement value file; F108: peak time file; 200: index value calculation unit; S201: RRI calculation process; S202: SD calculation process; S203: RM calculation process; S204: SD/RM calculation process; S205: correlation calculation unit calling process; F206: RRI file; F207: SD file; F208: RM file; F209: SD/RM file; 300: correlation calculation unit; S301: moment correlation coefficient calculation process; S302: mental stress determination unit calling process; F303: correlation coefficient file; 400: mental stress determination unit; S401: correlation coefficient evaluation process; S402: threshold value determination process; S403: display ON process; S404: display OFF process; S405: outgoing notification process; 12: memory; 13: display; 20: pulse wave measurement device; 21: LED; 22: photo transistor; 23: amplifier; 24: AD converter; 25: pulse wave signal; 30: another system; 41: ear lobe; 42: finger; 51: graph; 52: table

1-8. (canceled)

9. A mental stress detection device comprising:
processing circuitry to:
calculate a first index value, the first index value being an index of an activity state of parasympathetic nerves with elapse of time, and a second index value, the second index value being an index of an activity state of sympathetic nerves with elapse of time, based on a plurality of heartbeat intervals RRI, wherein the processing circuitry calculates a standard deviation SD of the plurality of heartbeat intervals RRI and a root mean square RM of differences RD between heartbeat intervals RRI and calculates a ratio SD/RM between the standard deviation SD and the root mean square RM; and
calculate a time corresponding correlation, the time corresponding correlation being a correlation between the first index value and the second index value and being a correlation associated with time, wherein the processing circuitry calculates a correlation between the root mean square RM and the ratio SD/RM as the time corresponding correlation.

10. The mental stress detection device according to claim 9, wherein the processing circuitry determines a level of mental stress based on the time corresponding correlation.

11. The mental stress detection device according to claim 10, wherein:
a value of the time corresponding correlation is determined with respect to time, and
the processing circuitry determines a level of mental stress through comparison between the value of the time corresponding correlation and a threshold value.

12. The mental stress detection device according to claim 10, wherein
the processing circuitry determines a period which does not include a negative correlation in the time corresponding correlation as a period in which mental stress is higher than in other periods.

13. The mental stress detection device according to claim 9, wherein the processing circuitry calculates the first index value and the second index value based on the plurality of heartbeat intervals RRI by using Fourier transformation.

14. The mental stress detection device according to claim 10, wherein the processing circuitry outputs at least one of a determination result and a time corresponding correlation.

15. A non-transitory computer-readable medium storing a mental stress detection program that causes a computer to execute:
a process of calculating a first index value, the first index value being an index of an activity state of parasympathetic nerves with elapse of time, and a second index value, the second index value being an index of an activity state of sympathetic nerves with elapse of time, based on a plurality of heartbeat intervals RRI, wherein the process of calculating the first index value and the second index value calculates a standard deviation SD of the plurality of heartbeat intervals RRI and a root mean square RM of differences RD between heartbeat intervals RRI and calculates a ratio SD/RM between the standard deviation SD and the root mean square RM; and
a process of calculating a time corresponding correlation, the time corresponding correlation being a correlation between the first index value and the second index value and being a correlation associated with time, wherein the process of calculating the time corresponding correlation calculates a correlation between the root mean square RM and the ratio SD/RM as the time corresponding correlation.

16. The mental stress detection device according to claim 11, wherein
the processing circuitry determines a period which does not include a negative correlation in the time corresponding correlation as a period in which mental stress is higher than in other periods.

17. The mental stress detection device according to claim 11, wherein
the processing circuitry outputs at least one of a determination result and a time corresponding correlation.

18. The mental stress detection device according to claim 12, wherein
the processing circuitry outputs at least one of a determination result and a time corresponding correlation.

19. The mental stress detection device according to claim 16, wherein
the processing circuitry outputs at least one of a determination result and a time corresponding correlation.
